(19) United States
(12) Patent Application Publication
(10) Pub. No.: US 2005/0059001 A1
(43) Pub. Date: Mar. 17, 2005

(54) Nucleic acids isolated in neuroblastoma

(76) Inventor: Akira Nakagawara, Chiba (JP)

Correspondence Address: FITCH EVEN TABIN AND FLANNERY, 120 SOUTH LA SALLE STREET, SUITE 1600, CHICAGO, IL, 60603-3406, US

(21) Appl. No.: 10/479,081

(22) PCT Filed: May 30, 2002
(86) PCT No.: PCT/JP02/05295

(30) Foreign Application Priority Data
May 31, 2001 (JP) 2001-163666
Aug. 24, 2001 (JP) 2001-255260

(51) Int. Cl. C12Q 1/68; C07H 21/04; C12N 9/64

(52) U.S. Cl. 435/6; 435/69.1; 435/320.1; 435/226; 435/325; 536/23.2

(57) ABSTRACT

There are disclosed a nucleic acid whose expression is enhanced in human neuroblastoma with favorable prognosis based on comparison between human neuroblastoma with favorable prognosis and human neuroblastoma with unfavorable prognosis, the nucleic acid comprising any one of base sequences set forth in SEQ ID NO:1 to NO:366 in the Sequence Listing, a nucleic acid comprising a portion of any of those base sequences, and an isolated nucleic acid capable of hybridizing to a complementary base sequence of the foregoing under stringent conditions. It discloses gene sequences relating to favorable or unfavorable prognosis of neuroblastoma and will enable the provision of their genetic information and the diagnosis of favorable or unfavorable prognosis.

### TECHNICAL FIELD

This invention relates to nucleic acids whose expression is enhanced in human neuroblastoma with favorable prognosis based on comparison between human neuroblastoma with favorable prognosis and human neuroblastoma with unfavorable prognosis.

### BACKGROUND ART

(Tumorgenesis and Genes)

Individual tumors exhibit distinct characteristic natures, and their biological properties are not necessarily identical even though the basic principle of oncogenesis is the same. Rapid advances in the understanding of cancer from a molecular biological and molecular genetic perspective in recent years have opened the way to an explanation of oncogenesis and tumor cell biology on the genetic level.

(Neuroblastomas)

Neuroblastoma is a pediatric cancer occurring in sympathetic gangliocytes and adrenal medullary cells which originate from cells of the peripheral sympathetic nervous system. Of these sympathetic nervous system cells, neural crest cells in the initial stage of development migrate to the abdomen, differentiating and maturing at sites where sympathetic ganglia are formed. Some of these cells migrate further to the adrenal bodies, penetrating through the adrenal cortex which is already in the process of formation, and reaching the medulla and forming medullary substance there. The neural crest cells also serve as a source of other peripheral nerve cells, differentiating into dorsal root ganglia (sensory nerves), skin pigment cells, thyroid C cells, some pulmonary cells, intestinal gangliocytes, and the like.

(Prognosis for Neuroblastoma)

Neuroblastoma is characterized by a varied clinical profile (Nakagawara, Shinkeigashu no Hassei to Sono Bunshi Kiko [Neuroblastoma Development and Molecular Mechanism], Shoni Naika 30, 143, 1998). For example, neuroblastomas occurring at less than one year of age have very favorable prognosis, with the majority undergoing differentiation and cell death, and spontaneous regression. Currently, most neuroblastomas discovered by a positive result in the commonly performed mass screening of 6-month-old infant urine are of the type which tend to undergo this spontaneous regression. On the other hand, neuroblastomas occurring at age 1 or higher are highly malignant and lead to death of the infant in the majority of cases. It is also hypothesized that a somatic mutation occurs in highly malignant neuroblastomas in infants older than one year of age, which are of monoclonal nature, whereas in naturally regressing neuroblastomas, the genetic mutation remains at only a germline mutation. See Knudson A G, et al.: Regression of neuroblastoma IV-S: A genetic hypothesis, N. Engl. J. Med. 302, 1254 (1980)).

(Genes which allow the diagnosis of prognosis for neuroblastoma)

With recent advances in molecular biology research, it has become clear that expression of the high affinity nerve growth factor (NGF) receptor TrkA is closely connected with control of differentiation and cell death. See Nakagawara A., The NGF story and neuroblastoma, Med. Pediatr. Oncol., 31, 113 (1998). Trk is a membrane-spanning receptor, existing as the three main types, Trk-A, -B and -C. These Trk family receptors play an important role in specific nerve cell differentiation and survival in the central nervous and peripheral nervous systems. See Nakagawara, et al., Shinkeigasaiboushu ni Okeru Neurotrophin Juyoutai no Hatsugen to Yogo [Expression of Neurotrophin Receptors and Prognosis in Neuroblastoma], Shoni Geka (Pediatric Surgery), 29:425-432, 1997. The survival and differentiation of tumor cells is controlled by signals from Trk tyrosine kinase and Ret tyrosine kinase. In particular, the role of TrkA receptor is most significant, with TrkA expression being notably high in neuroblastomas with favorable prognosis, and its signals exerting a powerful control over survival and differentiation of tumor cells, and cell death (apoptosis). In neuroblastomas with unfavorable prognosis, on the other hand, TrkA expression is significantly suppressed, while tumor development is aided by a mechanism in which survival is promoted by signals from TrkB and Ret.

It has become clear that amplification of the neural oncogene N-myc has become clearly associated with the prognosis of neuroblastoma. See Nakagawara, Nou-shinkeishuyo no Tadankai Hatsugan [Multistage Oncogenesis of Cerebral and Neural Tumors], Molecular Medicine, 364, 366(1999). This gene, first cloned in neuroblastoma, is ordinarily only present in a single copy per haploid set in normal cells and neuroblastomas with favorable prognosis, whereas it has been found to be amplified several dozen times in neuroblastomas with unfavorable prognosis.

Up till the present time, however, no oncogene other than N-myc is known to be expressed in neuroblastomas, and absolutely no genetic information other than that of N-myc has been known in relation to favorable or unfavorable prognosis.

### DISCLOSURE OF THE INVENTION

This invention has been accomplished in light of the circumstances described above, and its object is to identify the gene sequences which are related to favorable or unfavorable prognosis of neuroblastoma, and to allow the provision of their genetic information as well as the diagnosis for favorable or unfavorable prognosis.

As a result of conducting diligent research, the present inventors have examined the prognoses of neuroblastomas and have succeeded in constructing cDNA libraries from both clinical tissues with favorable prognosis and with unfavorable prognosis. Approximately 2400 clones were respectively obtained from these two types of cDNA libraries and were classified according to the prognosis of neuroblastoma (whether favorable or unfavorable).

Moreover, the present inventors found that the expression of a considerable number of the genes is enhanced only in clinical tissues of neuroblastoma with favorable prognosis among the classified genes.

Based on such knowledge, the present inventors have succeeded in providing base sequence information for the detection and cloning of the genes only expressed in clinical tissues with favorable prognosis.

Furthermore, based on the aforementioned base sequence information on the regions it has been made possible to carry out the method for detection of prognosis and to design tumor markers which can be used therefor, and this invention has thereupon been completed.

Specifically, this invention aims at providing a nucleic acid whose expression is enhanced in human neuroblastoma with favorable prognosis based on comparison between human neuroblastoma with favorable prognosis and human neuroblastoma with unfavorable prognosis, the nucleic acid comprising any one of base sequences set forth in SEQ ID NO:1 to NO:366 in the Sequence Listing as well as providing a nucleic acid comprising a portion of the base sequence. This invention also provides an isolated nucleic acid characterized in that it hybridizes to the nucleic acid described above or its complementary nucleic acid under stringent conditions.

The nucleic acids of this invention are those whose expression is enhanced in human neuroblastoma with favorable prognosis based on comparison between human neuroblastoma with favorable prognosis and human neuroblastoma with unfavorable prognosis and those nucleic acids are characterized by their capabilities of being used in the diagnosis for prognosis of human neuroblastoma. The preferred nucleic acid for this purpose is a nucleic acid comprising any one of base sequences set forth in SEQ ID NO:121, SEQ ID NO:125, SEQ ID NO:132, SEQ ID NO:133, SEQ ID NO:165, SEQ ID NO:171, SEQ ID NO:187, SEQ ID NO:188, SEQ ID NO:210, SEQ ID 229, SEQ ID NO:232, SEQ ID NO:273 and SEQ ID NO:281 in the Sequence Listing or a nucleic acid related thereto (e.g., a nucleic acid comprising a portion of the base sequence). The particularly preferred nucleic acid is a nucleic acid comprising any one of base sequences set forth in SEQ ID NO:121, SEQ ID NO:125, SEQ ID NO:132, ID NO:165, SEQ ID NO:171, SEQ ID NO:187, SEQ ID NO:188, SEQ ID 229, SEQ ID NO:232, SEQ ID NO:273 and SEQ ID NO:281 in the Sequence Listing or a nucleic acid related thereto (e.g., a nucleic acid comprising a portion of the base sequence).

This invention also provides a diagnostic agent for the detection of a neurological disease characterized by containing at least one nucleic acid comprising a portion or the whole of the base sequence set forth in SEQ ID NO:1 to SEQ ID NO:366 in the Sequence Listing. Specifically, such tumor detection diagnostic agents include DNA chips and microarrays both of which are produced using the above-mentioned nucleic acids, for example. Accordingly, this invention also provides a microarray composition characterized by containing plural numbers of nucleic acids comprising a portion or the whole of the base sequence set forth in SEQ ID NO:1 to SEQ ID NO:366 in the Sequence Listing. Preferably, such composition comprises all the nucleic acids, i.e., a total of 366 nucleic acids each of which comprises a portion or the whole of a base sequence set forth in SEQ ID NO:1 to SEQ ID NO:366 in the Sequence Listing. More preferably, the composition is that which contains a total of 13 nucleic acids each of which comprises a portion or the whole of a base sequence set forth in SEQ ID NO:121, SEQ ID NO:125, SEQ ID NO:132, SEQ ID NO:133, SEQ ID NO:165, SEQ ID NO:171, SEQ ID NO:187, SEQ ID NO:188, SEQ ID NO:210, SEQ ID NO:229, SEQ ID NO:232, SEQ ID NO:273 and SEQ ID NO:281 in the Sequence Listing.

Further according to this invention there is provided an isolated nucleic acid which is DNA characterized by hybridizing to the nucleic acid mentioned above or to its complementary nucleic acid under stringent conditions. There is also provided a diagnostic kit for the prognosis of human neuroblastoma containing as the effective ingredient, a primer set comprising a pair of those nucleic acids (DNA).

In addition, this invention provides a method of diagnosing the prognosis of human neuroblastoma, the method comprising detecting the presence or absence of a nucleic acid comprising any one of base sequences set forth in SEQ ID NO:1 to NO:366 in the Sequence Listing from a clinical tissue sample of the neuroblastoma.

### BRIEF DESCRIPTION OF THE DRAWINGS

FIG. 1 is a figure corresponding to an electrophoregram showing an example of the results of determination of the gene expression levels in human neuroblastomas with favorable prognosis and with unfavorable prognosis by semi-quantitative PCR.

FIG. 2 is a figure corresponding to electron micrographs showing a primary culture containing SCG neurons of a new born mouse.

### BEST MODE FOR CARRYING OUT THE INVENTION

The term “nucleic acid(s)” as used in this invention refers to, for example, DNA or RNA, or polynucleotides derived therefrom which are active as DNA or RNA, and preferably they are DNA or RNA.

The term “hybridize under stringent conditions” means that two nucleic acid fragments hybridize to each other under the hybridization conditions described by Sambrook, J. et al. in “Expression of cloned genes in E. coli”, Molecular Cloning: A Laboratory Manual (1989), Cold Spring Harbor Laboratory Press, New York, USA, 9.47-9.62 and 11.45-11.61.

More specifically, the “stringent conditions” refers to hybridization at approximately 45° C., 6.0×SSC, followed by washing at 50° C., 2.0×SSC. The stringency may be selected by choosing a salt concentration in the washing step from approximately 2.0×SSC, 50° C. as low stringency to approximately 0.2×SSC, 50° C. as high stringency. Also, the temperature in the washing step may be increased from room temperature, or approximately 22° C. as low stringency conditions, to approximately 65° C. as high stringency conditions.

The term “isolated nucleic acid(s)” as used in the present specification refers to a nucleic acid or a polynucleotide containing substantially no cellular substances or culture medium, if prepared by recombinant DNA techniques, or containing substantially no precursor chemical substances or other chemical substances, if prepared by chemical synthesis.

The term “favorable prognosis” as used in the present specification refers to a condition of human neuroblastoma in which the tumor is localized or has become a regressing or benign sympathetic ganglion neoplasm, and is judged to have low malignancy based on N-myc or other tumor markers. According to a preferred embodiment of the invention, a favorable prognosis is a case of stage 1 or 2, with an onset age of less than one year and survival without recurrence for 5 or more years after surgery, and with no noted amplification of N-myc in the clinical tissue; however, there is no limitation to such specific cases. The term “unfavorable prognosis” as used in the present specification refers to a condition of human neuroblastoma in which progression of the tumor has been observed, and it is judged to have high malignancy based on N-myc or other tumor markers. According to a preferred embodiment of the invention, an unfavorable prognosis is a case of stage 4, with an onset age of greater than one year, death within 3 years after surgery and noted amplification of N-myc in the clinical tissue; however, there is no limitation to such specific cases.

The nucleic acids of this invention have been found in the clinical tissues of human neuroblastoma and such nucleic acids have the characteristics described below.

Neuroblastoma is a tumor consisting of actual nerve cells, of which only two types of tumor are known in humans, and analysis of the genes expressed therein is expected to provide very useful knowledge for understanding the biology of nerve cells. Specifically, it is extremely difficult, and practically impossible, to obtain site-specific homogeneous tissue from the brain or peripheral nerves. On the other hand, a neuroblastoma consists of an almost homogeneous nerve cell population (though tumorized) derived from peripheral sympathetic nerve cells, and thus offers a high possibility of obtaining homogeneous expression of neuro-related genes. Furthermore, since neuroblastoma is a type of cancer, it will characteristically have many important genes expressed in the immature stage of neurogenesis.

Clinically and biologically, neuroblastoma can be neatly classified into favorable prognosis and unfavorable prognosis types. Cancer cells from neuroblastoma with favorable prognosis are characterized by having a very slow rate of proliferation, with spontaneous regression beginning at some point. Findings to date have confirmed that nerve cell differentiation and apoptosis (nerve cell death) occur in the spontaneous regression, and that the differentiation which occurs in the maturation stages of normal nerve cells and programmed cell death are phenomena very closely resembling each other. Consequently, it is highly probable that the analysis of genes expressed in such tumors will lead to obtaining important information relating to nerve cell differentiation and apoptosis.

Neuroblastomas with unfavorable prognosis are tumors consisting of cancer cells which continue to exhibit definitely malignant proliferation. The probability is very high, therefore, that they have a large number of important genes connected with nerve cell proliferation or genes expressed in undifferentiated nerve cells. In other words, it is highly probable that these will allow the obtainment of genetic information completely different from the profile of genes expressed in neuroblastomas with favorable prognosis.

It is commonly reported that nerve cells contain more expressed gene types than cells derived from other organs. Neuroblastoma cell lines are derived from clinical tissues with unfavorable prognosis, and it is believed that the gene expression profile in the case of tumor development and progression is substantially altered from that of normal nerve cells.

Neuroblastoma is characteristically a pediatric tumor, and because of the very low possibility of effects by acquired factors, it is expected that analysis of the mechanism of cancerization will also yield embryological information with high probability. More surprisingly, the genes or the gene fragments according to this invention include genes whose expression is enhanced only in specific cell cycle phases, and this further suggests the very strong possibility of obtaining genetic information highly useful for the analysis of cancerization mechanisms and related to development and differentiation.

The nucleic acids, having the characteristics mentioned above and from which the aforementioned information can be obtained, are available from human neuroblastoma clinical tissues and have any of the base sequences set forth in SEQ ID NO:1 to NO:366 in the Sequence Listing, or a portion thereof.

As a result of comparing gene expression levels in clinical tissues from human neuroblastomas with favorable prognosis and with unfavorable prognosis, a highly significant difference was found in each of the nucleic acids having base sequences set forth in SEQ ID NO:1 to NO:366 in the Sequence Listing. That is, the expression of these nucleic acids was enhanced in human neuroblastomas with favorable prognosis. Thus, in addition to providing the useful genetic information described above, the base sequences set forth in SEQ ID NO:1 to NO:366 can also be utilized as data for tumor markers to diagnose favorable or unfavorable prognosis of neuroblastoma, by detecting DNA and/or RNA having any of these base sequences.

Specifically, this invention will make it possible to obtain various forms of diagnosis for prognosis on or relating to human neuroblastoma through the following means.

(1) Probes for Hybridization

The nucleic acid comprising a portion or the whole of a base sequence disclosed in this invention (which may be referred to as “nucleic acid(s) of the invention”) may be at least used as a probe for hybridization in order to detect genes expressed in human neuroblastoma. The nucleic acids of the invention may also be used as probes for hybridization in order to determine gene expression in various tumors and normal tissues, to identify the distribution of the gene expression.

When the nucleic acid comprising a portion or the whole of a base sequence disclosed in this invention is used as a probe for hybridization, there are no particular limitations on the actual method of hybridization. As preferred methods there may be mentioned, for example, Northern hybridization, Southern hybridization, colony hybridization, dot hybridization, fluorescence in situ hybridization (FISH), in situ hybridization (ISH), DNA chip methods, and microarray methods.

As one application example of the hybridization, the nucleic acid of this invention may be used as a probe for Northern hybridization to measure the length of mRNA or to quantitatively detect gene expression in an assayed sample.

When the nucleic acid of the invention is used as a probe for Southern hybridization, it enables the detection of the presence or absence of the base sequence in genomic DNA of an assayed sample.

The nucleic acid comprising a portion or the whole of a base sequence disclosed in this invention can also be used as a probe for fluorescence in situ hybridization (FISH) to identify the location of the gene on a chromosome.

The nucleic acid of the invention can also be used as a probe for in situ hybridization (ISH) to identify the tissue distribution of gene expression.

When the nucleic acid of the invention is used as a probe for hybridization, a nucleic acid residue length of at least 40 is necessary; and among the gene sequences according to the invention, a nucleic acid having 40 or more contiguous residues is preferably used. More preferably, one having 60 or more nucleic acid residues is used.

Nucleic acid probe techniques are well known to one skilled in the art, and for example, conditions suitable for hybridization between a probe of specific length according to the invention and the target polynucleotide may be readily determined. In order to obtain hybridization conditions optimal to probes of various lengths, Sambrook et al. “Molecular Cloning: A Laboratory Manual, 2nd Edition, Cold Spring Harbor (1989) may be followed for such manipulations which are well known to one skilled in the art.

The probe of the invention is preferably labeled in an easily detectable fashion. The detectable label may be any type or portion which can be detected either visually or using devices. As commonly used detectable labels there may be mentioned radioactive isotopes such as ³²P, ¹⁴C, ¹²⁵I, ³H and ³⁵S. Biotin-labeled nucleotides may be incorporated into DNA or RNA by nick translation, chemical or enzymatic means. The biotin-labeled probes are detected after hybridization using labeling means such as avidin/streptavidin, fluorescent labels, enzymes, gold colloidal complexes or the like. The nucleic acid may also be labeled by binding with a protein. Nucleic acid cross-linked to a radioactive or fluorescent histone single-stranded DNA binding protein may also be used.

(2) Primers for Use in PCR

For other possible methods of detecting genes, DNA that is the nucleic acid comprising a portion or the whole of a base sequence disclosed in this invention can be used as a primer in a polymerase chain reaction (PCR). For example, RNA may be extracted from a sample to be assayed, and the gene expression can be semi-quantitatively measured by RT-PCR. This may be carried out by a method well known to one skilled in the art. For example, “Molecular Cloning: A Laboratory Manual,” (T. Maniatis, Cold Spring Harbor Laboratory Press) or Idenshibyo Nyumon [Introduction to Genetic Diseases] (Takahisa, S.: Nankodo Publishing) may be followed.

When the DNA that is a nucleic acid of the invention is used as a PCR primer, a base length of 10 to 60 is necessary; and among the gene sequences according to the invention, the nucleic acid having 10 to 60 contiguous bases is preferably used. More preferably, one having 15 to 30 bases is used. Generally, a primer sequence with a GC content of 40-60% is preferred. Also, there is preferably no difference in the Tm values of the two primers used for amplification. Preferably there is no annealing at the 3′ ends of the primers and no secondary structure is formed in the primers.

(3) Nucleic Acid Screening

The nucleic acid comprising a portion or the whole of a base sequence disclosed in this invention can also be used to detect the expression distribution of a gene which is expressed in various tissues or cells. This can be accomplished, for example, by using the nucleic acid of the invention as a probe for hybridization or as a primer for PCR.

Expression distribution of a gene can also be detected using a DNA chip, microarray or the like. That is, the nucleic acid of the invention may be directly attached to the chip or array. RNA extracted from cells of a tissue may be labeled there with a fluorescent substance or the like, hybridized, and an analysis can be made of the type of tissue cells with high expression of the gene. The DNA attached to the chip or array may be the reaction product of PCR using the nucleic acid of the invention. U.S. Pat. No. 5,605,662 to Heller et al. describes an example of the method for attaching nucleic acids to a chip or array.

By utilizing the aforementioned technology, it is possible to use as a diagnostic agent, at least one among the nucleic acids comprising portions or the whole of base sequences disclosed in this invention. In recent years, it has becoming clear that the genetic information possessed by individuals governs their susceptibility to a certain disease as well as governs the effectiveness of a particular drug. The DNA chips or microarrays produced using the aforementioned nucleic acids are used to clarify the cause-effect relationship between the disease of a subject and the nucleic acid, which will enable not only the diagnosis of that disease but also the selection of a drug to be administered. Specifically, if the result detected using the DNA chip or microarray is employed as an indicator of selection of a drug to be administered, the expression level of one nucleic acid among the nucleic acids of the invention can be examined but also the expression levels of two or more nucleic acids can be comparatively examined to select the drug to be administered, which will then enable more accurate judgment. As used herein, the disease is not particularly limited insofar as it is that which can be diagnosed by the nucleic acids of this invention. Preferably, the disease is a neurological disease and more preferably, neuroblastoma.

(4) DNA Cloning

The nucleic acid comprising a portion or the whole of a base sequence disclosed in this invention can be at least used for cloning a gene which is expressed in human neuroblastoma. For example, by using the nucleic acid of the invention as a probe for Northern hybridization or Southern hybridization, or as a primer for PCR, cloning of a gene containing the nucleic acid comprising a portion or the whole of a base sequence disclosed in this invention is possible. As the genes capable of being subjected to cloning there may be mentioned genes with differing levels of expression particularly between neuroblastoma with favorable prognosis and neuroblastoma with unfavorable prognosis, genes whose forms of expression differ in other tissues or cancer cells, genes whose expression is cell cycle phase-dependent, genes induced upon neurodifferentiation and genes whose expression is regulated by oncogenes or tumor suppressor genes.

(5) Methods of Diagnosing Tumor Prognosis and Tumor Markers to be Used Therefor

The nucleic acid comprising a portion or the whole of a base sequence disclosed in this invention can be used as a probe for hybridization, or as a primer for PCR to determine the presence or absence of enhancement in the gene expression in sample cells, which enables the identification of prognosis. To determine the presence or absence of enhancement in the gene expression, any method that utilizes nucleic acids (probes) capable of hybridizing to the nucleic acid comprising any given sequence of the base sequence disclosed in this invention is provided for use. Prognosis can be diagnosed as favorable if the amount of nucleic acid hybridizing to the probe is increased in the sample cell. When the nucleic acid is used as a primer for PCR, RNA is extracted from the sample to be assayed and the gene expression can be semi-quantitatively measured by the RT-PCR method.

(6) Antisense Oligonucleotides

According to another embodiment of this invention there are provided antisense oligonucleotides having the base sequences according to the invention. As will be considered in practicing this invention, the antisense oligonucleotides may readily be prepared such that they can bind to RNA corresponding to the base sequence of this invention and can thereby inhibit the synthesis of RNA.

(7) Gene Therapy

According to a further embodiment of this invention, there are provided therapeutic genes to be used in gene therapy. As will be considered in practicing this invention, the gene according to this invention may be transferred into a vector for use in gene transportation, whereby the transgene can be expressed by an arbitrary expression promoter and can be used for the gene therapy of cancers, for example.

1. Vectors

The transferable viral vectors may be prepared from DNA viruses or RNA viruses. They may be any viral vector of an MoMLV vector, a herpes virus vector, an Adenovirus vector, an AAV vector, a HIV vector, a Seidai virus vector and the like. One or more proteins among the constituent protein group of a viral vector are substituted by the constituent proteins of a different species of virus, or alternatively a part of the base sequence constituting genetic information is substituted by the base sequence of a different species of virus to form a viral vector of the pseudo-type which can also be used in this invention. For example, there is mentioned a pseudo-type viral vector wherein the Env protein (an envelop protein of HIV) is substituted by the VSV-G protein (an envelop protein of vesicular stomatitis virus or VSV) (Naldini L., et al., Science 272, 263-267, 1996). Further, virues having a host spectrum other than human is usable as the viral vector insofar as they are efficacious. As for the vectors other than those of viral origin, there may be used complexes of calcium phosphate and nucleic acid, ribosomes, cation-lipid complexes, Seidai virus liposomes, polymer carriers having polycation as the main chain and others. In addition, methods such as electroporation and gene guns may be used as a gene transfer system.

2. Expression Promoters

As for the expression cassettes to be used for the therapeutic gene, any cassettes without any particular limitations may be used insofar as they can cause genes to express in the target cells. One skilled in the art can readily select such expression cassettes. Preferably, they are expression cassettes capable of gene expression in the cells derived from an animal, more preferably, expression cassettes capable of gene expression in the cells derived from a mammal, and most preferably expression cassettes capable of gene expression in the cells derived from a human. The gene promoters that can be used as expression cassettes include: for example, virus-derived promoters from an Adenovirus, a cytomegalovirus, a human immunodeficiency virus, a simian virus 40, a Rous sarcoma virus, a herpes simplex virus, a murine leukemia virus, a sinbis virus, a hepatitis type A virus, a hepatitis type B virus, a hepatitis type C virus, a papilloma virus, a human T cell leukemia virus, an influenza virus, a Japanese encephalitis virus, a JC virus, parbovirus B19, a poliovirus, and the like; mammal-derived promoters such as albumin, SR α, a heat shock protein, and an elongation factor; chimera type promoters such as a CAG promoter; and the promoters whose expression can be induced by tetracyclines, steroids and the like.

### EXAMPLES

This invention will now be explained in greater detail by way of the examples and production examples; however, the invention will not be restricted to those example.

### Production Example 1

### Construction of cDNA Library from Human Neuroblastoma

1. Obtaining Samples

Human neuroblastoma clinical tissue specimens were quasi-aseptically frozen immediately after surgical extraction and then preserved at −80° C.

2. Selecting Samples with Favorable Prognosis

Prognosis of the samples obtained in 1. above was carried out based on the following criteria.

Favorable prognosis:
Stage 1 or 2
Age of onset less than one year
Survival for longer than 5 years after surgery without recurrence
No amplification of N-myc

Unfavorable prognosis:
Stage 4
Age of onset older than 4 years
Death within 3 years after surgery
Amplification of N-myc

Amplification of N-myc in the aforementioned sample types was confirmed in the following manner.

The clinical tissue sample obtained in 1. above was thinly sliced with a scalpel and then thoroughly homogenized after addition of 5 ml of TEN buffer (50 mM Tris-HCl (pH=8.0)/1 mM EDTA/100 mM NaCl). Upon adding 750 μl of SDS (10%) and 125 u 1 of proteinase K (20 mg/ml) to the mixture, it was gently stirred and allowed to stand at 50° C. for 8 hours. This was followed by phenol/chloroform treatment and finally ethanol precipitation to obtain purified genomic DNA. A 5 μg portion of the obtained genomic DNA was completely digested with the restriction endonuclease EcoRI (NEB Inc.), and an N-myc probe was used to determine amplification of N-myc by Southern hybridization.

3. Preparation of mRNA from Clinical Tissue of Human Neuroblastoma with Favorable Prognosis

A 2-3 g portion of the clinical tissue sample of human neuroblastoma judged to have favorable prognosis in 2. above was treated using a Total RNA Extraction Kit (QIAGEN Inc.) and the total RNA was extracted. The extracted total RNA was purified using an oligo dT cellulose column (Collaborative Research, Inc.) to obtain a pool of mRNA with a polyA structure.

4. Dephosphorylation of mRNA

A 100-200 μg portion of the mRNA pool prepared in 3. above was dissolved in 67.3 μl of distilled sterile water containing 0.1% diethyl pyrocarbonate (DEPC), and then 20 μl of 5XBAP buffer (Tris-HCl (500 mM, pH=7.0)/mercaptoethanol (50 mM)), 2.7 A 1 of RNasin (40 unit/μl: Promega Inc.) and 10 μl of BAP (0.25 unit/μl, bacteria-derived alkali phosphatase: Takara Shuzo Co. Ltd.) were added. The mixture was reacted at 37° C. for 1 hour to effect dephosphorylation of the 5′ end of the mRNA. This was followed by phenol/chloroform treatment two times, and finally ethanol precipitation to obtain a purified dephosphorylated mRNA pool.

5. Decapping of Dephosphorylated mRNA

The total amount of the dephosphorylated mRNA pool prepared in 4. above was dissolved in 75.3 μl of distilled sterile water containing 0.1% DEPC, and then 20 μl of 5×TAP buffer (sodium acetate (250 mM, pH=5.5)/mercaptoethanol (50 mM), EDTA (5 mM, pH=8.0)), 2.7 μl of RNasin (40 unit/μl) and 2 μl of TAP (tobacco acid pyrophosphatase: 20 unit/μl) were added. The mixture was reacted at 37° C. for 1 hour to effect decapping treatment of the 5′ end of the dephosphorylated mRNA. The dephosphorylated mRNA of incomplete length with no capped structure remained without decapping, and with the 5′ end dephosphorylated. This was followed by phenol/chloroform treatment and ethanol precipitation to obtain a purified decapped mRNA pool.

6. Preparation of Oligo-Capped mRNA

The total amount of the decapped mRNA pool prepared in 5. above was dissolved in 11 g 1 of distilled sterile water containing 0.1% DEPC, and then 4 μl of 5′-oligo RNA (5′-AGCAUCGAGUCGGCCUUGGCCUACUGG-3′: 100 ng/μl), 10 μl of 10× ligation buffer (Tris-HCl (500 mM, pH=7.0)/mercaptoethanol (100 mM)), 10 μl of magnesium chloride (50 mM), 2.5 μl of ATP (24 mM), 2.5 μl of RNasin (40 unit/μl), 10 μl of T4 RNA ligase (25 unit/μl: Takara Shuzo Co. Ltd.) and 50 μl of polyethylene glycol (50% w/v, PEG8000: Sigma Corporation) were added. The mixture was reacted at 20° C. for 3 hours for ligation of the 5′-oligo RNA to the 5′ end of the decapped mRNA. The dephosphorylated mRNA of incomplete length with no capped structure resulted in no ligation to the 5′-oligo RNA. This was followed by phenol/chloroform treatment and ethanol precipitation to obtain a purified oligo-capped mRNA pool.

7. Removal of DNA from Oligo-Capped mRNA

The oligo-capped mRNA pool prepared in 6. above was dissolved in 70.3 μl of distilled sterile water containing 0.1% DEPC, and then 4 A 1 of Tris-HCl (1 M, pH=7.0), 5.0 μl of DTT (0.1 M), 16 p 1 of magnesium chloride (50 mM), 2.7 μl of RNasin (40 unit/μl) and 2 μl of DNaseI (5 unit/1: Takara Shuzo Co. Ltd.) were added. The mixture was reacted at 37° C. for 10 minutes to dissolve the excess DNA. This was followed by phenol/chloroform treatment and ethanol precipitation and column purification (S-400HR: Pharmacia Biotech Inc.), to obtain a purified DNA(−) oligo-capped mRNA pool.

8. Preparation of 1st Strand cDNA

The DNA(−) oligo-capped mRNA pool prepared in 7. above was reverse transcribed using SuperScript II (kit by Life Tech Oriental, Inc.) to obtain a pool of 1st strand cDNA. The pool of DNA(−) oligo-capped mRNA was dissolved in 21 μl of sterile distilled water, and then 10 μl of 10×First Strand buffer (kit accessory), 8 μl of DNTP mix (5 mM, kit accessory), 6 μl of DTT (0.1 M, kit accessory), 2.5 μl of oligo-dT adapter primer (5 pmol/μl, 5′-GCGGCTGAAGACGGCCTATGTGGCC TTTTTTTTTTTTTTTTT-3′), 2.0 μl of RNasin (40 unit/μl) and 2 μl of SuperScript II RTase (kit accessory) were added. The mixture was reacted at 42° C. for 3 hours to effect reverse transcription. This was followed by phenol/chloroform treatment, alkali treatment and neutralization treatment to dissolve all the RNA and purification was carried out by ethanol precipitation.

9. Preparation of 2nd Strand cDNA

The 1st strand cDNA pool prepared in 8. above was subjected to PCR amplification using Gene Amp (kit by Perkin Elmer Inc.). The pool of 1st strand cDNA was dissolved in 52.4 μl of sterile distilled water, and then 30 μl of 3.3× Reaction buffer (kit accessory), 8 μl of DNTP mix (2.5 mM, kit accessory), 4.4 μl of magnesium acetate (25 mM, kit accessory), 1.6 μl of Primer F (10 pmol/μl, 5′-AGCATCGAGTCGGCCTTGTTG-3′), 1.6 μl of Primer R (10 pmol/μl, 5′-GCGCTGAAGACGGCCTATGT-3′) and 2 μl of rTth (kit accessory) were added. A 100 μl portion of mineral oil was gently added to the mixture and overlayed thereon. After denaturing the reaction solution at 94° C. for 5 minutes, a cycle of 94° C. for 1 minute, 52° C. for 1 minute and 72° C. for 10 minutes was repeated 12 times, and then the solution was allowed to stand at 72° C. for 10 minutes to complete the PCR reaction. This was followed by phenol/chloroform treatment and ethanol precipitation to obtain a 2nd strand cDNA pool.

10. SfiI Treatment of 2nd Strand cDNA

The 2nd strand cDNA pool prepared in 9. above was dissolved in 87 μl of sterile distilled water, and then 10XNEB buffer (NEB Inc.), 100XBSA (bovine serum albumin available from NEB Inc.) and 2 μl of SfiI (restriction endonuclease, 20 unit/μl, NEB Inc.) were added. The mixture was reacted overnight at 50° C. to effect SfiI restriction endonuclease treatment. This was followed by phenol/chloroform treatment and ethanol precipitation to obtain a pool of cDNA which had been SfiI-treated at both ends.

11. Size Fractionation of SfiI-Treated cDNA

The SfiI-treated cDNA pool prepared in 10. above was electrophoresed on 1% agarose gel and a fraction with >2 kb was purified using Geneclean II (Bio101 Inc.). The purified cDNA pool was dissolved in 100 μl of sterile distilled water and allowed to stand at 37° C. for 6 hours. This was followed by phenol/chloroform treatment and ethanol precipitation to obtain a long-chain cDNA pool.

12. Preparation of cDNA Library

The long-chain cDNA pool prepared in 11. above was ligated into the cloning vector pME18S-FL3 (provided by Prof. Sumio Kanno of the Institute of Medical Science, Tokyo University) using a DNA Ligation Kit ver.1 (kit by Takara Shuzo Co. Ltd.). The long-chain cDNA pool was dissolved in 8 μl of sterile distilled water, and then 1 μl of pME18S-FL3 pretreated with restriction endonuclease DraIII, 80 μl of Solution A (kit accessory) and 10 μl of Solution B (kit accessory) were added and reaction was conducted at 16° C. for 3 hours. This was followed by phenol/chloroform treatment and ethanol precipitation for purification to obtain a cDNA library.

### Example 1

### Transformation into E. coli

The cDNA library prepared in Production Example 1 above was used for transformation into E. coli (TOP-10: Invitrogen Corporation). The cDNA library was dissolved in 10 μl of sterile distilled water and mixed with TOP-10. The mixture was then incubated on ice for 30 minutes, at 40° C. for 1 minute and on ice for 5 minutes. After adding 500 μl of SOB medium, shake culturing was performed at 37° C. for 60 minutes. Appropriate amounts thereof were seeded onto ampicillin-containing agar media and culturing was continued at 37° C. for a day and a night to obtain E. coli clones.

The E. coli clones on agar media obtained were collected with toothpick and suspended in 120 μl of LB medium prepared in a 96-well plate. The 96-well plate was then allowed to stand overnight at 37° C. for culturing of the E. coli. A 72 μl portion of 60% glycerol solution was then added and preserved at −20° C. (glycerol stock).

### Example 2

### Base Sequence Determination

1. Preparation of Plasmid

The 10 μl of glycerol stock prepared in Example 1 was transferred to a 15 ml centrifugation tube, and then 3 ml of LB medium and 50 μg/ml of ampicillin were added and shaking was carried out overnight at 37° C. for culturing of the E. coli. A QIAprep Spin Miniprep Kit (QIAGEN Inc.) was then used to extract and purify a plasmid DNA from the E. coli.

2. Analysis of Both End Sequences

Both end sequences of the plasmid DNA prepared in 1. above were determined using a DNA Sequencing Kit (kit by ABI). There were combined 600 ng of plasmid DNA, 8 μl of premix (kit accessory) and 3.2 pmol of primers, and sterile distilled water was added to a total of 20 μl. After denaturing the mixture at 96° C. for 2 minutes, a cycle of 96° C. for 10 seconds, 50° C. for 5 seconds and 60° C. for 4 minutes was repeated 25 times for reaction. The product was then purified by ethanol precipitation. Sequencing was carried out by polyacrylamide gel electrophoresis under denaturing conditions, using ABI377 (ABI).

### Example 3

### Homology Search Using Database

An Internet-mediated DNA sequence homology search was conducted for the samples on which the both end-sequences were analyzed in Example 2. The homology search was conducted using the BLAST of the NCBI (National Center of Biotechnology Information, USA).

### Example 4

### Determination of Gene Expression Levels in Human Neuroblastomas with Favorable Prognosis and Unfavorable Prognosis by Semi-Quantitative PCR

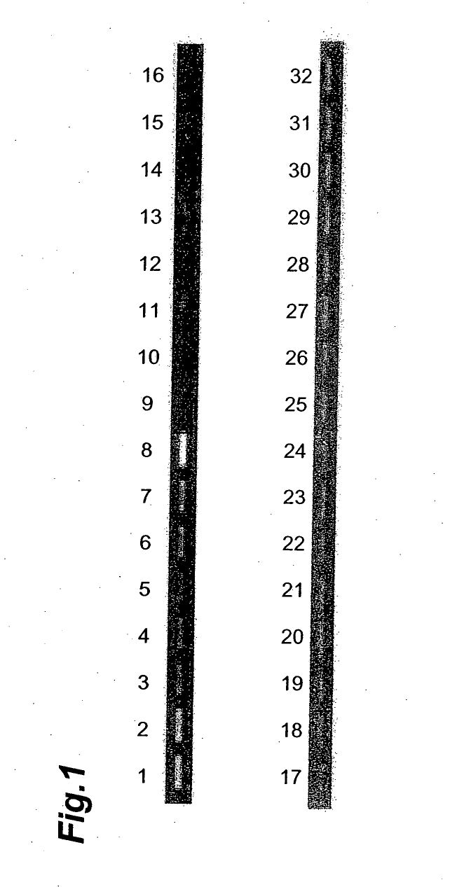

PCR primers were synthesized from portions of the genes obtained in Example 3, and the expression levels in the clinical tissues of human neuroblastomas with favorable prognosis and unfavorable prognosis were comparatively quantitated. mRNA was extracted from the human neuroblastoma clinical tissues by the method described in Examples 3, and rTaq (Takara Shuzo Co. Ltd.) was used for PCR reaction. Specifically, 5 μl of sterile distilled water, 2 μl of mRNA, 1 μl of 10XrTaq buffer, 1 μl of 2 mM dNTPs, 0.5 μl each of the synthesized primer set and 0.5 μl of rTaq were combined. After denaturing the mixture at 95° C. for 2 minutes, a cycle of 95° C. for 15 seconds, 55° C. for 15 seconds and 72° C. for 20 seconds was repeated 35 times, and then the mixture was allowed to stand at 72° C. for 6 minutes for PCR reaction. The reaction solution was subjected to 1% agarose gel electrophoresis. One example of the results of determination of the gene expression levels in human neuroblastomas with favorable prognosis and unfavorable prognosis by semi-quantitative PCR is shown in FIG. 1. Here, the explanations of the respective lanes are as follows:
Lanes 1-8: expression of nbla-4183 (SEQ ID NO:154, SEQ ID NO:155 in the Sequence Listing) in a clinical sample of neuroblastoma with favorable prognosis;
Lanes 9-16: expression of nbla-4183 (SEQ ID NO:154, SEQ ID NO:155 in the Sequence Listing) in a clinical sample of neuroblastoma with unfavorable prognosis;
Lanes 17-24: expression of GAPDH in a clinical sample of neuroblastoma with favorable prognosis; and
Lanes 25-32: expression of GAPDH in a clinical sample of neuroblastoma with unfavorable prognosis.

Consequently, nucleic acids whose expression is enhanced only in human neuroblastoma with favorable prognosis have been identified in the base sequences set forth in SEQ ID NO:1 to SEQ ID NO:366.

### Example 5

### Gene Group with Varying Gene Expression Levels by NGF Regulated Apoptosis in Murine Superior Cervical Gangliocytes

It is known that NGF (nerve growth factor) exerts protective action against apoptosis in human neuroblastoma induced by an antimitotic agent (such as neocarzinostatin). (Cortazzo M H, J. Neurosci, 16, 3895-3899 (1996)). This action is believed to be due to the inhibition of apoptosis by the binding of NGF to p75. It has also been pointed out that the regression of neuroblastoma (causing favorable prognosis) at clinical stage is connected to the apoptosis of the tumor cells.

In order to identify the apoptosis-associated genes in neuroblastoma, the gene group with varying gene expression levels by NGF regulated apoptosis in murine superior cervical gangliocytes was examined.

Superior cervical gangliocytes (SCG neurons) were isolated from C57BL/6J mice (0 to 1 day of age) in 7 to 10 animals. Seven wells of cells were arranged to be grown on a 24-well cell culture plate at about 1 to about 2×10⁴ cells per well. RNA was further collected from about 1 to about 2×10⁴ cells (Day 1 as control).

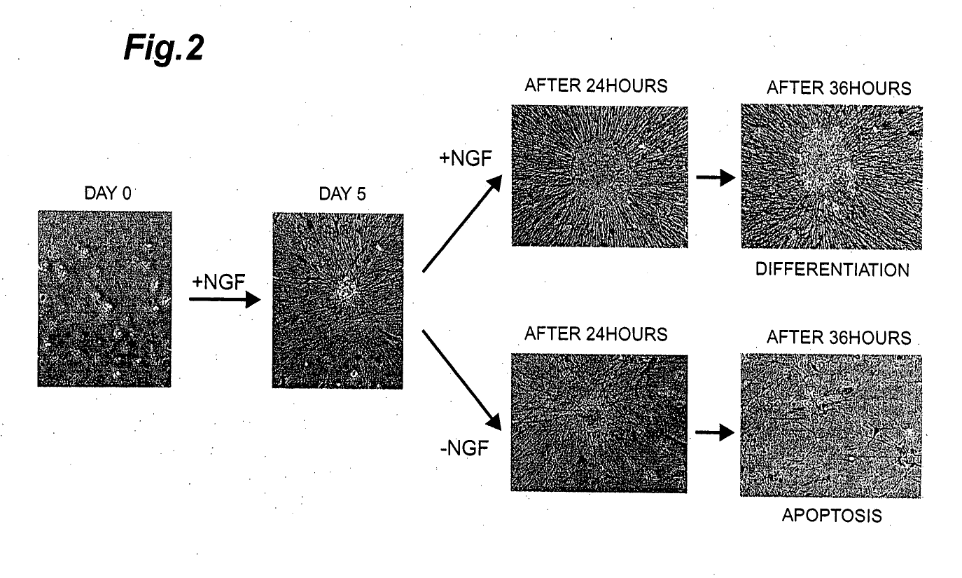

After the culture obtained by adding NGF (5 mg/ml) to the 7-well cells was incubated for. 5 days, RNA was collected from one-well cells (+NGF; after 5 days) Cultures in three wells each of the 6 wells were exchanged with cultures with addition of NGF (5 mg/ml) or anti-NGF antibodies (1%), and they were respectively incubated for 12 hours, 24 hours and 48 hours. Using portions of the samples, cells were examined under an electron microscope to identify the morphological differences between the case where NGF had been added and the case where anti-NGF antibodies had been added. The results are shown in FIG. 2. NGF induced differentiation was observed in the cells with NGF addition, whereas apoptosis was observed in the cells with anti-NGF antibody addition. As a result of NGF depletion by the anti-NGF antibodies, it was apparent that apoptosis was induced. RNA was collected from the respective well cells after 12 hr, 24 hr, and 48 hr culturing. These samples were denoted +NGF 12 hrs, +NGF 24 hrs, +NGF 48 hrs, −NGF 12 hrs, −NGF 24 hrs, and −NGF 48 hrs.

As described above, RNAs of the respectively treated cells from approximately 160 mice were concentrated by ethanol precipitation and cDNAs were synthesized from 1 μg RNAs using reverse transcriptase. These cDNAs were used as templates and gene expression levels were compared by PCR. The primers used were constructed from the homologous genes in mice based on the genes found in human neuroblastoma cells. The base sequences of the primers used for the comparison of expression are summarized in Table 1 together with the gene identification numbers (ID) for the targeted genes for comparison.

TABLE 1

| | | | |
|---|---|---|---|
| primer | primer sequence | primer | primer sequence |

| | | | |
|---|---|---|---|
| nbla00031m-F3 | TCAGAAGGCTTCGAGACTG | nbla00031m-R3 | GCAGATATCTTGTCAAAGGT |
| nbla00100m-f | GGTGAGCCCTAACATCCACA | nbla00100m-r | AGCCCGTAAGCCATCAATC |
| nbla00115m-F3 | ATCATGGTGAAAGGCACGTC | nbla00115m-R3 | CATAATTTCCACGTGTTGTG |
| nbla00116m-F3 | AGAGGTTAGCCCTGAGACAG | nbla00116m-R3 | TCTATGCTTCCAGCAGGTAC |
| nbla00124m-F3 | CCTTTATATTAGAACGTGGC | nbla00124m-R3 | TAGGTTTTCAGGACTTGG |
| nbla00144m-F | GCAGGATAGCAACCTTGACA | nbla00144m-R | ATGCATGCTTTCGTGTGTTC |
| nbla00150m-2f | CACCCTGGAGAAACACAAACTC | nbla00150m-2r | ATGCCGTCCATCTTCATCAC |
| nbla00170m-F3 | AACATGAATCTGTGGGTGAC | nbla00170m-R3 | TCAATCGCTTGACCTTCCTC |
| nbla00204m-F3 | AAGCATTGAAGTAACAACCG | nbla00204m-R3 | TGCATTTGACATATGAGAGC |
| nbla00225m-F2 | TATTAGAACGTGGCCCTCCA | nbla00225m-R2 | TCGGGACTAGCAGGACAGAA |
| nbla00301m-F3 | TAAACCAGCAACCCTAACAG | nbla00301m-R3 | ACTAGAATCAGACCTGCCTT |
| nbla00315m-F | AACTGGCAGCAAAGAAGGTC | nbla00315m-R | AGCAGAAGCACGTCAGTAAGG |
| nbla00402m-F2 | TCACCCAGAATGAGACATGG | nbla00402m-R2 | GTCAGAAGGTGACACGGTGA |
| nbla00433m-F3 | CCCAAGGAGGTAAGAGCCTG | nbla00433m-R3 | AAGCGCTGGAGCTTGTCGGT |
| nbla00437m-F3 | AGAACGGTGTAATTCAGAGA | nbla00437m-R3 | GTCATCAGCAAGCTCGAATA |
| nbla00537m-F3 | TTTATATTAGAACGTGGCCC | nbla00537m-R3 | AGGACTTGGTAGCTTCTCGG |
| nbla00551m-F | AATTTTAAAAAGGCAGGATATACAAC | nbla00551m-R | AAGGAGAGGTAGCATTTTATGTGC |
| ori00660m-F | GGCATCCTCATCTCCTCAGA | ori00660m-R | TCTGAAGCAGTTCGAAGCAC |
| nbla00761m-f | TGGTTCAAACTCTTCCCTCCT | nbla00761m-r | CTTCCACCTGTGCGTTTTCT |
| nbla00831m-F3 | TGGGAAATCCACCTGCATC | nbla00831m-R3 | ACCAGCTCACCCTGAATGTG |
| nbla00908m-2f | CGTGGCAACACCTTTTTATTC | nbla00908m-2r | ACACGGCATGGGTTTGTT |
| nbla02874m-f | TGAGGAGTTGAATGCTGACCT | nbla02874m-r | CATCGGGGTTAATGCTCTTG |
| nbla03086m-f | ACTTGCTCATGGTGCTGTCTC | nbla03086m-r | CGCAGAGCCTGGTAATCTTC |
| nbla03113m-F | CCTCAAGAACCAGACCAAGC | nbla03113m-R | CTGATGCTGAGGAGCTGACA |
| nbla03199m-f | GGCCACCAGAGAGGTAATG | nbla03199m-r | GTGCTGACCTAAGACCCAAAG |
| nbla03267m-f | CTGCCTTTGAGATGGTGATG | nbla03267m-r | TGTAGTGCTTTGCATTGTTGG |
| nbla03646m-f | TACCTCTTCGGCTGGATGG | nbla03646m-r | GCTTGGGCAGGATGAATG |
| nbla03755m-F | AGCCGATGAAGTGTCTGCTT | nbla03755m-R | RAGCCACAAAAGCAGGTTAGG |
| nbla03771m-f | GCAGCAGATATAGGGACACAGA | nbla03771m-r | CACGCATAAATGGCTACACC |
| nbla03777m-F3 | AGATTATTCACCTGTAAGC | nbla03777m-R3 | TTTTCCACATGTCCAGCACC |
| nbla03831m-f | GTGACCAGCAAGGAAACACA | nbla03831m-r | GAGGATTCAGCCACGAACA |
| nbla03862m-F | GCATGGAGGAAACCATTAGG | nbla03862m-R | TGGCTCTTCAACCAAACCTT |
| nbla04021m-F2 | ATGACCTGGCACTAGGCTTG | nbla04021m-R2 | AACACATCTGCTGGCTTCTG |
| nbla04137m-f | ACGACGACACTGACAACCAC | nbla04137m-r | GCTTGACCTCCGACTCATCT |
| nbla04196m-f | CACCCATCTGTGTCTGTGGT | nbla04196m-r | CGTGCTGACGATGATGTTG |
| nbla04261m-2f | GTGTTTGGCACTACATCACCA | nbla04261m-2r | CCGGGTTTTCCATTTTCAC |
| nbla04300m-f | TACAGTGGGAACTGCGTTTG | nbla04300m-r | CAGGGTTCGTATGCAGGAG |
| nbla10058m-f | AGCCATTACAGGTGGCAAGA | nbla10058m-r | GTTGGGTCGATCTTAGGAGGTAG |
| nbla10070m-F3 | TAACAACTCTAGCACCATC | nbla10070m-R3 | TACTTAGCAGAACAGAAGAG |
| nbla10071m-f | GTGGACACCAAGAATGCAAG | nbla10071m-r | CAGCCAACTGTGGTAAGAAGG |
| nbla10120m-F | CAGTGCAGCCTTGGAAGTGT | nbla10120m-R | TCAAAAGCTGCGTGTGTCTC |
| nbla10143m-F3 | TTAGTGAGTACACGAGCTGG | nbla10143m-R3 | ACTTAACCCAGACTGACCAC |
| nbla10283m-F3 | AGATGTTTAAGGGCAAACC | nbla10283m-R3 | TGGAGCCTCTTGGATCTC |
| nbla10300m-F3 | AACATCCTGGTGGAACAGC | nbla10300m-R3 | CTCTATAGTAACGACCAAAC |
| nbla10314m-f | GCTTGAGGACAGTGAAAACCA | nbla10314m-r | TGGAGTGAGAGGATGGGAAG |
| nbla10317m-2f | GCAGGGGACACAGGACTCTAC | nbla10317m-2r | AAGCTCCTTCTGGCTCAACA |
| nbla10329m-F | CCGAGATCTTCTGCCTTCAT | nbla10329m-R | TCCTTGCCGTCTCAAACTCT |
| nbla10363m-F3 | ATCTCTCTAGTGCCATGAC | nbla10363m-R3 | AGTCTTGCTAAGACTTTCAG |
| nbla10383m-f | CAGTGCGGTTGTGGTCTATCT | nbla10383m-r | TGAGGCGTTGACTTTCTGG |
| nbla10388m-F | TTCAGCAGGTCCTAGCCAAG | nbla10388m-R | RTGGAAGCTGCTGAAGAAACA |
| nbla10457m-F | GCCTTTCTTTGTCTGGAACG | nbla10457m-R | GGGTGAAGCAATTTCACAGG |
| nbla10485m-F | AAATGGCAGTTTGACTGTGG | nbla10485m-R | TTGGCTGAGTTCTCCCTCAT |
| nbla10527m-F3 | ATTAATCCTGCACTCTTACG | nbla10527m-R3 | AGTTCCATTTCTACAGCAAG |
| nbla10535m-F | TTGGTCGTGAGGTGGATTCT | nbla10535m-R | ATCTTGCCAGCCACAGACTT |
| nbla10545m-F | AGAGTCACCTGCGACCCTTA | nbla10545m-R | AGCTCTAGCAGCCAGCACAT |
| nbla10677m-f | CATAATCTTCTCCGGCTTCATC | nbla10677m-r | GTCTGGTATTTCCGTGAGGTTT |
| nbla10687m-F3 | ATCTCCCATCGACTCACTGC | nbla10687m-R3 | TGGCTTTACTGGTCATACAG |
| nbla10696m-f | TGATTCTCCAAGGCAAGGT | nbla10696m-r | GATTTCCCCATTGACTGCT |
| nbla10988m-2f | AGCCTTTGCTACCCTCTTGC | nbla10988m-2r | GGCGAAACACTCCTCTCGT |
| nbla11030m-F3 | AACCTCGTAAAAACCATGGC | nbla11030m-R3 | AGCAGTGACTTGAGCATTTG |
| nbla11042m-2f | CGACACCTCTCATTGCACAC | nbla11042m-2r | TCCGTCTCAAATCCACACAC |
| nbla11051m-f | AGCACAATTCCCCAGACAC | nbla11051m-r | CTGTAGCCCTTACTGTTTGACC |
| nbla11189m-F | CTGTGTTCTGATGCCAATGC | nbla11189m-R | TGCAACTTTCTCCACCAAGA |
| nbla11589m-F2 | GGAGCTAGCCAAGATGATCG | nbla11589m-R2 | CTGGCCATCCTAGAGGAGAA |
| nbla11882m-F | AATTTTAAAAAGGCAGGATATACAAC | nbla11882m-R | AAGGAGAGGTAGCATTTTATGTGC |
| nbla11895m-F3 | AATCTTCCTCCCAACCCATG | nbla11895m-R3 | TGACCCTGCTGAAGGAAGCG |
| nbla20001m-F2 | CCCTGAATGTTGAACGAGTG | nbla20001m-R2 | TGCACATTGAAGAGGCAAAC |
| nbla20019m-F1 | CCGAGGTCAACATTTGTTCC | nbla20019m-R1 | GAGCAGAGCACAGACAGTGG |
| nbla20125m-F1 | CTGGAATCATCCAGGCTTTG | nbla20125m-R1 | GCGTCCAGGATAACAGCACT |
| nbla20134m-F1 | GTCTTCAAGCAGCGACAGTG | nbla20134m-R1 | CCTTCAGGGTCTGGTTGATG |
| nbla20146m-F1 | GGGACCACTAACCAGCTGAA | nbla20146m-R1 | AAATGTCTGACCCCTCCTCA |
| nbla20181m-F1 | CCAGGATGGAGTAGCCAAGA | nbla20181m-R1 | GCCAGTGATCTCCAGGTTTG |
| nbla20182m-F1 | ACTGGGGAGGAATGGCTAGT | nbla20182m-R1 | CTGGCTGGAGGAAAAAGGAC |
| nbla20211m-F1 | CTGAGGTGCTGATGATCCTG | nbla20211m-R1 | CCAAACTCCTGCTCTTCTCG |
| nbla20231m-F1 | AGCAGTTTGGTGCTGTTGGT | nbla20231m-R1 | GCTTGTTCCATGGTTGGACT |
| nbla20250m-F2 | ACGAGGACCACAGGACTCAA | nbla20250m-R2 | TGGGCTCCATAGTTTGTTCC |
| nbla20268m-F1 | ATTCTCTCTGGAGGCGATGA | nbla20268m-R1 | TTTCCTCACAGCTCCCTCTC |
| nbla20378m-F2 | TTTAGCTGTTTGCAAATAAGATGT | nbla20378m-R2 | AATGCAGTGTGTGGGATGTG |
| nbla20421m-F1 | TCCGACATGATGGTTCTCCT | nbla20421m-R1 | AGATCCAGGAGTCACCCAAA |
| nbla20487m-F3 | CACAGGTGTCAAAGCACGTT | nbla20487m-R3 | TGTAGCACTCGCTGTTGCTC |
| nbla20511m-F1 | TCCTGAAGCCTTCTTGCCTA | nbla20511m-R1 | GCTTGTGGCAACCAGAAAGT |
| nbla20541m-F1 | AGGTGGGAGTCGACCTTTCT | nbla20541m-R1 | GCATCCTTCAACTTGGTCCT |
| nbla20638m-F1 | GCAGCAAGGAAGAGGACAAG | nbla20638m-R1 | AGCAATCTTCGTCTGGGAAG |
| nbla20688m-F1 | AGAAGGACCTCCTCCCAAAG | nbla20688m-R1 | TGCAACAGTCCTCTTCCTTG |
| nbla20709m-F2 | ATTAGTTGGGACCTGCCTTG | nbla20709m-R2 | GCCCATTTTCTTCAGCAGAG |
| nbla20765m-F1 | AGAGCAGCTCAGCTACCACA | nbla20765m-R1 | GAAGGCAACTTTGGTGTTGG |
| nbla20798m-F1 | AAACGGGTACAGGATGGAGA | nbla20798m-R1 | CAACTGGAGGTCGGAGGATA |
| nbla20874m-F1 | TGGTCTGTTTAACAATTGACCTG | nbla20874m-R1 | GCTGCATCTTCCAACATTCTT |
| nbla20968m-F1 | CCGTGCAGTTTGACATGAAT | nbla20968m-R1 | TGTGGCATCTCATTCAGCAT |
| nbla21013m-F1 | CACCCTTTGAAGTGACGGTA | nbla21013m-R1 | CTGAAAAACCAGCCCACACT |
| nbla21024m-F1 | CTCTGTGGGATGACACATGC | nbla21024m-R1 | TTTGGCACCTTGTCATTTTG |
| nbla21077fm-F1 | CCTGCTTAAGAGTAAGCCTGGT | nbla21077fm-R1 | AGTTCTGTGGGCTATAGGATCG |
| nbla21130m-F1 | TGTGTCTTCCGACTTTCTGG | nbla21130m-R1 | TCTTTCCCTGAGTGCTTGGT |
| nbla21189m-F1 | AGAGGTGGAAGCACTTCAGG | nbla21189m-R1 | AAAGTCCTTGGTGCTCTCGT |
| nbla21233m-F1 | CTGGAGCCTGTTTGATGGTT | nbla21233m-R1 | TGCTTTCCTTACTGGCAGGT |
| nbla21266m-F1 | GAACATGGGCACTGACTGG | nbla21266m-R1 | CATCAGGGCTAGGAGACTCG |
| nbla21297m-F3 | CAGCAATGAATCCTCCAATGT | nbla21297m-R3 | TGGTGGTTCTCCCTGTGATT |
| nbla21298m-F2 | AGACCAGTGGCTCGTCAAAC | nbla21298m-R2 | TTGCAGACATCAGGGGTGT |
| nbla21337m-F2 | TCTGCACCAGAGAATCCACA | nbla21337m-R2 | ATAGGGCTTTTCTCCCGTGT |
| nbla21367m-F1 | CAGAAAAACGGTGGAGGACT | nbla21367m-R1 | CCCTGCCTTGTTCTCCATAA |
| nbla21375m-F1 | CAAGCATGCAGGAAGAACTC | nbla21375m-R1 | AATGTTCGTAGCCGATCCAG |
| nbla21413m-F1 | TGTGTTTGCTGGGGAGTATG | nbla21413m-R1 | TCATTTGCAGCCACTCTACG |
| nbla21569m-F1 | TGGAGCTCAGAAGAGGAGGA | nbla21569m-R1 | CGCAACATGAAGTCCATCAG |
| nbla21681m-F1 | CAAAGTCCCTCTCCTTCAGC | nbla21681m-R1 | CCCTAGTGGCCAACTCTGAT |
| nbla21761m-F1 | TTACCCAGGTGGTTCAGCAT | nbla21761m-R1 | TACCCATCAGGGTGATGACA |
| nbla21843m-F1 | GCCAGGAAGTGAGGAATGAG | nbla21843m-R1 | CCCGGATGACCTGAATGTAG |
| nbla21855m-F1 | AACACACTGGCGTTCATCTG | nbla21855m-R1 | GACTCCCACTTGCGTCTCTG |
| nbla21922m-F1 | GACATGGAAGGCATGCTGTA | nbla21922m-R1 | GACAGACGCTTCAGCGAAAT |
| nbla21934m-F1 | AGCCTTGGTGCTGAAGATGT | nbla21934m-R1 | CTTATGTGACCGTGCACCTG |
| nbla21936m-F1 | CGCCTGACAATCTCAATTCC | nbla21936m-R1 | CTCACGTGTCTGCGTTTGAT |
| nbla21950m-F1 | AGGGGATCCGGAAGTATGAC | nbla21950m-R1 | AAGCCGCTCATCTGGTAGAG |
| nbla22027m-F1 | TGAGAAAACGGTCCTGAAGC | nbla22027m-R1 | TGTCAGACCCTTGGCATCTT |
| nbla22028m-F1 | TACCTGAGTCGGACACGATG | nbla22028m-R1 | GAGAGCCAGACAAGCTTTGG |
| nbla22093m-F1 | ACAAGGACCCCTGTGCTAAC | nbla22093m-R1 | TGAGGACAGTGGCAGGTGTA |
| nbla22153m-F1 | TGCGAGGATTAACTCCAAGG | nbla22153m-R1 | GGCAACTTTGGCTGAAGAGT |
| nbla22182m-F2 | AGTCCCCATTGGGATACTGC | nbla22182m-R2 | TGGGCAATAATTTGGAAACC |
| nbla22218m-F1 | AGCCACACTGTTAGCAGCAA | nbla22218m-R1 | CGAATGTCCAGAAGGGAGAG |
| nbla22228m-F1 | CAGCCTGTGAATGGTGTGAA | nbla22228m-R1 | TGAGGGAAGCTGTGGAAGAG |
| nbla22298m-F1 | TGACATCTGCTTGTCCTTGG | nbla22298m-R1 | GGACGGCAGTACCAAGAGTG |
| nbla22344m-F2 | GAGTCTGCCATTGGCTTTGT | nbla22344m-R2 | CTGCTTGCTCTGTTCCACTG |
| nbla22351m-F1 | AGACAGGGTCTGGCTGTGTT | nbla22351m-R1 | TGAGGCCAGGAGTTCAAGAC |
| nbla22352m-F1 | CTGGCACAATGTCTTCACAA | nbla22352m-R1 | TTGAAAGGGGAGATTCCTGA |
| nbla22361m-F1 | CGACAGATGACCTTGATTCG | nbla22361m-R1 | GCCTCAGAAGCCTTTCTCAA |
| nbla22382m-F1 | GTTGGCGAGCTAGCAAAACT | nbla22382m-R1 | TGCCATCCTTCTCACAGATG |
| nbla22394m-F1 | TTACCCATAATGCCCTCCAC | nbla223944m-R1 | CAAAACGACAGCAGCAGAAC |
| nbla22451m-F1 | GGCATTGGAGGTTGTCATTC | nbla22451m-R1 | GCTTGCTCTTCACCAGGAAC |
| nbla22455m-F1 | GAAGACCCTGGTTTTTGCAG | nbla22455m-R1 | CAACTCGGTTGGTGAAATCAG |
| nbla22465m-F1 | AGGAGAAGCCTCATCAACCA | nbla22465m-R1 | TCAGGCAGACATTCCCAGAT |
| nbla22474m-F1 | AATGTGCCGGTTTTCTCATC | nbla22474m-R1 | CCAACCCCTTAGACATGCAC |
| nbla22549m-F1 | TCTGTGAAAGGGCATGTGAG | nbla22549m-R1 | GGAGGGATTTCATTGCTCTG |
| nbla22704m-F1 | TCTCAGTTGGGTTTGGAAGC | nbla22704m-R1 | GGAGAAAAGCCAGAGTGTCG |
| nbla22832m-F1 | CACGGGAACCCATTTGTATG | nbla22832m-R1 | GTGGAAGGAGCCGTTGATAA |
| nbla22886m-F1 | ATCGGCAGAGTGCATACCTT | nbla22886m-R1 | TCTGTGGCTCAATACGCTTG |
| nbla23020m-F1 | TTCCCACCAAATCAGTCTCC | nbla23020m-R1 | TGGCCACTGAAGTCTCAGGT |
| nbla23038m-F1 | ATGGTGAAAGTGGTTCAGTGC | nbla23038m-R1 | TGCATTTGCTGTGGATTACC |
| nbla23060m-F1 | GTGGCACAGTGGGAGCTATT | nbla23060m-R1 | GCCCAATCCTCTAAACAACG |
| nbla23218m-F1 | GTAAACAGCCCCTTGGTCAG | nbla23218m-R1 | ATGTGCAGTGTTCCCCACTT |
| nbla23394m-F1 | CAAGCGGTGGAGTACTTGAA | nbla23394m-R1 | CCTGCAGTTCCCAGTCTTTC |
| nbla23512m-F1 | CCCAGCCAGCCAAGTAGATA | nbla23512m-R1 | ACCCCACTCTTTGGGTCTCT |
| nbla23653m-F1 | ACTGGGCATCTGGATAGCAG | nbla23653m-R1 | CTTCGGAACCAGCCAACTTA |
| nbla23664m-F1 | TGAGGGGCTTCAAGCAGTAG | nbla23664m-R1 | GCACACTCACTTCCCAAGGA |
| nbla23666m-F1 | CGTGGTGGTGTGTATTTTGG | nbla23666m-R1 | GCGGTGACATAAAAGGCTGA |
| nbla23718m-F1 | ATGGAGAACTTGCCTGCACT | nbla23718m-R1 | TCAGCTTCACCCACACTTTG |
| nbla23719m-F1 | CTGTGGAGGAGTGGGATGTT | nbla23719m-R1 | GTTGCTTTTGGTTTCCCATC |
| nbla23760m-F1 | TGTGAGTCCTCCTGTTGTGG | nbla23760m-R1 | GAGCTGTCAAAATGGCTTCC |
| nbla23951m-F1 | TCACAGTTGCTGCCAAAGAG | nbla23951m-R1 | CGTCCCATTTTCCAGAGATG |
| nbla23973m-F1 | ACCAGTGTGAGGTGGTCAGA | nbla23973m-R1 | GTGGGAGGCCACAAACTTAG |
| nbla24082m-F1 | GGAGCAATCCAAGGAGATGA | nbla24082m-R1 | TGGACAGCCTCCTTCAGTTT |
| nbla24084m-F1 | GGTCGTTTAGGTGGCAAATG | nbla24084m-R1 | CCCAGGAATCTGCAAGGATA |
| nbla24104m-F1 | GTTGATGGAGCACCACAGAA | nbla24104m-R1 | CTGCATTGTTCAGCCAGGTT |
| nbla24131m-F1 | CACCAATGCTGTGAACTTGC | nbla24131m-R1 | TGACAGTCCAGCCTCACAGA |
| nbla24239m-F1 | TGGACACGCATAAGAAGCAG | nbla24239m-R1 | TGTCGAAGAAACTCCTGACG |
| nbla24285m-F1 | GTGGTGTGATGTCTGCCATT | nbla24285m-R1 | CCTTGTTGGACCTTGATTCC |
| nbla24297m-F1 | AAGGCTCTTTCCAGGAGGTC | nbla24297m-R1 | CCTTCAGGACACACAGGCTTA |
| nbla24348m-F1 | TAGTCCCCAAACCTGCTGTT | nbla24348m-R1 | GGCGAAGAAGAATTTGAACG |
| nbla24434m-F1 | GCCTGTGGTCAAAATCCAGT | nbla24434m-R1 | CTTTGCCTCAGTGGCTCTTC |
| nbla24460m-F1 | TTTCCAGGAGGTCAGGTTTG | nbla24460m-R1 | CCTTCAGGACACACAGGCTTA |
| nbla24468m-F1 | TGACAAAGACGCTGAACTGG | nbla24468m-R1 | GGGTTGTCAGAAAGCCTGAG |
| nbla24521m-F1 | TAATGGGAGTGCTGCCTCTG | nbla24521m-R1 | CATCTTTAAGGCTAACATGC |
| nbla24526m-F1 | AAGGAGCGCACCAACAGTAT | nbla24526m-R1 | ATAGGGTGGCTCAGGGAAAT |
| nbla24622m-F1 | GCACTTGGACCTCCCTTGTA | nbla24622m-R1 | GGTCCTGCACATTTCAACAG |
| nbla24672m-F1 | GGGCAGGACAATAAGACTGC | nbla24672m-R1 | TGTCCACAGCAGACACCCTA |
| nbla24686m-F1 | AAGAAGGCTCCGAGCTCAAT | nbla24686m-R1 | TCTGTGAGCTCCAGGCAGTA |
| nbla24709m-F1 | GCAGCATTCTGAGACACAGG | nbla24709m-R1 | GCTGGAGAGACCCAAGGACT |
| nbla24719m-F1 | CAGGGGATAACCTTCGTCAA | nbla24719m-R1 | AAGCAGCAACGTGGGATAAC |
| nbla24756m-F1 | ACATTGACAACCCTCCCAAG | nbla24756m-R1 | AAAGCAGCAGCCTCAGAGAA |
| nbla24831m-F1 | AAACGCTTGAGCTCTTCCAC | nbla24831m-R1 | AGCTCAGCAACCGCTCTAAA |
| nbla24893m-F1 | AGAGGGGCCAAGGGATATAA | nbla24893m-R1 | TACGAGGGCCTGTTTCAGAT |
| nbla24908m-F1 | TTCAGAGCATCGAGTTCACG | nbla24908m-R1 | TCATCCTCCTGGGTGAAGTT |
| nbla24972m-F1 | TCACCAAAACTGGCACAGAG | nbla24972m-R1 | TCGGACTGTGCTGCATTCTA |
| nbla24973m-F1 | CTGCACACCTCTCAATGCAG | nbla24973m-R1 | GAAGCAGCCCGATGTGTT |
| nbla24986m-F1 | CAGAGCAGTAACCGTGACCA | nbla24986m-R1 | ACCAGGACGGACACTGTTGT |

The results described below were obtained with the genes for which variations in the gene expression level were observed. The genes whose expression levels were decreased notably by NGF depletion (−NGF) were nbla-03646 (SEQ ID NO:121) (−NGF 12 hrs), nbla-03771 (SEQ ID NO:125) (−NGF 12 hrs), nbla-03831 (SEQ ID NO:132) (−NGF 24 hrs), nbla-04300 (SEQ ID NO:165)(−NGF 24 hrs), nbla-10120 (SEQ ID NO:171) (−NGF 48 hrs), nbla-10329 (SEQ ID NO:187) (−NGF 24 hrs), nbla-10363 (SEQ ID NO:188) (−NGF 12 hrs), nbla-10677 (SEQ ID NO:229)(−NGF 12 hrs), nbla-10696 (SEQ ID NO:232) (−NGF 12 hrs), nbla-11051 (SEQ ID NO:273) (−NGF 24 hrs), and nbla-11189 (SEQ ID NO:281)(−NGF 24 hrs). The genes whose expression levels were increased notably by NGF depletion (−NGF) was noted were nbla-03862 (SEQ ID NO:133) (−NGF 24 hrs) and nbla-10485 (SEQ ID NO:210) (−NGF 24 hrs) The gene whose expression level was increased notably by NGF addition was noted was nbla-10143 (SEQ ID NO:174)(+NGF).

The primer sets used to amplify those particular genes are shown in Table 2.

TABLE 2

| | | | |
|---|---|---|---|
| primer | primer sequence | primer | primer sequence |

| | | | |
|---|---|---|---|
| nbla03646m-f | TACCTCTTCGGCTGGATGG | nbla03646m-r | GCTTGGGCAGGATGAATG |
| nbla03771m-f | GCAGCAGATATAGGGACACAGA | nbla03771m-r | CACGCATAAATGGCTACACC |
| nbla03831m-f | GTGACCAGCAAGGAAACACA | nbla03831m-r | GAGGATTCAGCCACGAACA |
| nbla03862m-F | GCATGGAGGAAACCATTAGG | nbla03862m-R | TGGCTCTTCAACCAAACCTT |
| nbla04300m-f | TACAGTGGGAACTGCGTTTG | nbla04300m-f | CAGGGTTCGTATGCAGGAG |
| nbla10120m-F | CAGTGCAGCCTTGGAAGTGT | nbla10120m-R | TCAAAAGCTGCGTGTGTCTC |
| nbla10143m- | CAGTGCAGCCTTGGAAGTGT | nbla10143m- | TCAAAAGCTGCGTGTGTCTC |
| F3 | | R3 | |
| nbla10329m-F | CCGAGATCTTCTGCCTTCAT | nbla10329m-R | TCCTTGCCGTCTCAAACTCT |
| nbla10363m- | ATCTCTCTAGTGCCATGAC | nbla10363m- | AGTCTTGCTAAGACTTTCAG |
| F3 | | R3 | |
| nbla10485m-F | AAATGGCAGTTTGACTGTGG | nbla10485m-R | TTGGCTGAGTTCTCCCTCAT |
| nbla10677m-f | CATAATCTTCTCCGGCTTCATC | nbla10677m-r | GTCTGGTATTTCCGTGAGGTTT |
| nbla10696m-f | TGATTCTCCAAGGCAAGGT | nbla10696m-r | GATTTCCCCATTGACTGCT |
| nbla11051m-f | AGCACAATTCCCCAGACAC | nbla11051m-r | CTGTAGCCCTTACTGTTTGACC |
| nbla11189m-F | CTGTGTTCTGATGCCAATGC | nbla11189m-R | TGCAACTTTCTCCACCAAGA |

As described above, because the superior cervical gangliocytes are known to die (apoptosis) through NGF depletion, the genes that display variations in the expression level are closely connected to the mechanism of apoptosis.

### INDUSTRIAL APPLICABILITY

As described above, this invention discloses gene sequences relating to favorable or unfavorable prognosis of neuroblastoma and will enable the provision of their genetic information and the diagnosis of favorable or unfavorable prognosis.

1. A nucleic acid whose expression is enhanced in human neuroblastoma with favorable prognosis based on comparison between human neuroblastoma with favorable prognosis and human neuroblastoma with unfavorable prognosis, the nucleic acid comprising any one of base sequences set forth in SEQ ID NO:1 to NO:366 in the Sequence Listing.

2. The nucleic acid according to claim 1, wherein the base sequence is any one of base sequences set forth in SEQ ID NO:121, SEQ ID NO:125, SEQ ID NO:132, SEQ ID NO:133, SEQ ID NO:165, SEQ ID NO:171, SEQ ID NO:187, SEQ ID NO:188, SEQ ID NO:210, SEQ ID 229, SEQ ID NO:232, SEQ ID NO:273 and SEQ ID NO:281 in the sequence listing.

3. The nucleic acid according to claim 1, wherein the base sequence is any one of base sequences set forth in SEQ ID NO:121, SEQ ID NO:125, SEQ ID NO:132, ID NO:165, SEQ ID NO:171, SEQ ID NO:187, SEQ ID NO:188, SEQ ID 229, SEQ ID NO:232, SEQ ID NO:273 and SEQ ID NO:281 in the Sequence Listing.

4. A nucleic acid comprising a portion of any one of base sequences set forth in SEQ ID NO:1 to NO:366 in the Sequence Listing.

5. The nucleic acid according to claim 4, wherein the portion of the base sequence is derivable from any one of the base sequences set forth in SEQ ID NO:121, SEQ ID NO:125, SEQ ID NO:132, SEQ ID NO:133, SEQ ID NO:165, SEQ ID NO:171, SEQ ID NO:187, SEQ ID NO:188, SEQ ID NO:210, SEQ ID 229, SEQ ID NO:232, SEQ ID NO:273 and SEQ ID NO:281 in the Sequence Listing.

6. The nucleic acid according to claim 4, wherein the portion of the base sequence is derivable from any one of the base sequences set forth in SEQ ID NO:121, SEQ ID NO:125, SEQ ID NO:132, ID NO:165, SEQ ID NO:171, SEQ ID NO:187, SEQ ID NO:188, SEQ ID 229, SEQ ID NO:232, SEQ ID NO:273 and SEQ ID NO:281 in the Sequence Listing.

7. An isolated nucleic acid capable of hybridizing to the nucleic acid according to any of claims 1 to 4 or a complementary nucleic acid thereof.

8. A diagnostic agent for the detection of a neurological disease containing at least one nucleic acid comprising a portion or the whole of a base sequence set forth in SEQ ID NO:1 to SEQ ID NO:366 in the Sequence Listing.

9. The diagnostic agent for the detection of a neurological disease according to claim 8, wherein the base sequence is any one of the base sequences set forth in SEQ ID NO:121, SEQ ID NO:125, SEQ ID NO:132, SEQ ID NO:133, SEQ ID NO:165, SEQ ID NO:171, SEQ ID NO:187, SEQ ID NO:188, SEQ ID NO:210, SEQ ID 229, SEQ ID NO:232, SEQ ID NO:273 and SEQ ID NO:281 in the Sequence Listing.

10. The diagnostic agent for the detection of a neurological disease according to claim 8, wherein the base sequence is any one of the base sequences set forth in SEQ ID NO:121, SEQ ID NO:125, SEQ ID NO:132, ID NO:165, SEQ ID NO:171, SEQ ID NO:187, SEQ ID NO:188, SEQ ID 229, SEQ ID NO:232, SEQ ID NO:273 and SEQ ID NO:281 in the Sequence Listing.

11. The isolated nucleic acid according to claim 7, wherein the nucleic acid is DNA.

12. A diagnostic kit for the prognosis of human neuroblastoma containing as the effective ingredient, a primer set comprising a pair of the isolated nucleic acids according to claim 11.

13. A method of diagnosing the prognosis of human neuroblastoma, the method comprising detecting the presence or absence of a nucleic acid comprising any one of base sequences set forth in SEQ ID NO:1 to NO:366 in the Sequence Listing from a clinical tissue sample of the neuroblastoma.
